(19) United States
(12) Patent Application Publication
(10) Pub. No.: US 2011/0319724 A1
(43) Pub. Date: Dec. 29, 2011

(54) METHODS AND SYSTEMS FOR NON-INVASIVE, INTERNAL HEMORRHAGE DETECTION

(76) Inventor: Paul G. Cox, Huntsville, AL (US)

(21) Appl. No.: 12/665,811

(22) PCT Filed: Oct. 30, 2007
(86) PCT No.: PCT/US07/83042

§ 371 Date: Dec. 21, 2009

Related U.S. Application Data
(60) Provisional application No. 60/863,574, filed on Oct. 30, 2006.

(51) Int. Cl. A61B 5/00

(52) U.S. Cl. 600/301

(57) ABSTRACT

Methods and systems for detecting internal hemorrhaging in a person are provided. In an exemplary embodiment, one method includes the steps of measuring physiological conditions associated with the person and processing the measured physiological conditions using a probabilistic network to determine if the person has internal hemorrhaging. The method also includes the steps of determining the severity of any internal hemorrhaging by determining the amount of blood lost by the person and classifying this loss as non-specific, mild, moderate, and severe. The physiological measurements include an electrocardiogram, a photoplethysmogram, and oxygen saturation, respiratory, skin temperature, and blood pressure measurements. The probabilistic network included with one system determines whether there is internal hemorrhaging based on a number of factors including a physiological model, medical personnel inputs, transfer function, statistical, and spectral information, short and long term trends, and previous hemorrhage decisions.

### CROSS-REFERENCES TO RELATED APPLICATIONS

This application is a PCT application which claims benefit of co-pending U.S. Patent Application Ser. No. 60/863574, filed Oct. 30, 2006 and entitled “Automated, Non-Invasive, Internal Hemorrhage Detection,” which is hereby incorporated by reference.

A portion of the disclosure of this patent document contains material that is subject to copyright protection. The copyright owner has no objection to the facsimile reproduction by anyone of the patent document or the patent disclosure, as it appears in the U.S. Patent and Trademark Office patent file or records, but otherwise reserves all copyright rights whatsoever.

### BACKGROUND

1. Technical Field

The present invention relates generally to systems and methods for detecting internal hemorrhaging in a person.

More particularly, this invention pertains to a system and method for providing automated, real-time, non-invasive monitoring and detection of internal hemorrhaging in a person using non-invasive physiological measurements from the person and a probabilistic network which processes the physiological measurements to determine if there is internal hemorrhaging and, if so, the severity of that hemorrhaging.

2. Description of the Related Art

Shock is a serious medical condition where the tissue perfusion is insufficient to meet the required supply of oxygen and nutrients. Hypovolemic shock is the most common type of shock and occurs when there is insufficient circulating volume. Its primary cause is loss of fluid from circulation from either an internal or external source. An internal source may be hemorrhage. External causes may include extensive bleeding, high output fistulae or severe burns. Hypovolemic shock accounts for approximately 50% of the deaths on the battlefield and accounts for approximately 30% of the injured soldiers who die from wounds. In the civilian arena, hypovolemic shock is the leading cause of death from ages 1 to 44, and approximately 40% of patients suffering traumatic injuries die before they reach a hospital. Monitoring the onset of hypovolemic shock poses a major challenge because the body's compensatory mechanism buffers against the noticeable changes (in the early stage of shock) in the person's vital signs, thereby making it difficult to detect.

When a person loses a large quantity of blood, the arterial pressure decreases rapidly. This is followed by a series of compensatory cardiovascular responses that attempt to restore arterial pressure back to normal in order to sustain life. When the blood volume decreases, there will be a decrease in the venous return to the heart and a decrease in right arterial pressure. When the venous return decreases, there is a corresponding decrease in cardiac output. The decrease in cardiac output then leads to a decrease in arterial pressure.

The prior art teaches the use of various systems for monitoring physiological conditions associated with an injured person. For example, U.S. Pat. No. 7,079,888 is directed to a method and apparatus for monitoring the autonomic nervous systems of a person using non-stationary spectral analysis of the person's heart rate and respiratory signals. The apparatus and method uses real-time continuous wavelet transformation (CWT) in order to independently monitor the dynamic interactions between the sympathetic and parasympathetic divisions of the autonomic nervous system in the frequency domain. The apparatus and method described in the '888 patent allows spectral analysis to be applied to time-varying biological data, such as heart rate variability, respiratory activity, and blood pressure. The '888 patent does not describe a system and method that can be used to detect internal hemorrhaging in a person based on measured physiological conditions or the severity of that hemorrhaging.

A system for passively monitoring physiological conditions is described in U.S. Pat. No. 6,984,207. The system includes a piezoelectric film sensor made out of polyvinylidene fluoride that converts sensed physiological data into electrical signals, a band-pass filter for filtering out noise and isolating the signals, a pre-amplifier for amplifying the signals, and a computer for receiving and analyzing the signals and outputting data for real-time interactive display. The system detects mechanical, thermal and acoustic signatures reflecting cardiac output, cardiac function, internal bleeding, respiratory, pulse, apnea, and temperature. The signals are not standard vitals signs currently collected by physiological devices such as ECG, diastolic and systolic blood pressure, respiratory rate, SpO2, and PPG. Like the '888 patent discussed above, the '207 patent does not describe a system and method that can be used to detect internal hemorrhaging based on the measured physiological conditions of a person or the severity of that hemorrhaging.

A physiological sensing device is described in U.S. Pat. No. 6,491,647. This patent describes a non-invasive device for measuring physiological processes. Specifically, the '647 patent describes a device that can be applied externally to the body of an animal or human to detect and quantify displacement, force, motion, vibration and acoustic effects resulting from internal biological functions. Even more specifically, the '647 patent describes an inexpensive device that is compact, light, portable and comfortable, and operates satisfactorily even with imprecise location on the body, ambient noise, motion, and light. The device is designed to detect signals but not to analysis or interpret those signals. This patent, like the '207 and '888 patents discussed previously, does not describe a system and method that can be used to detect internal hemorrhaging based on the physiological conditions of a person or the severity of that hemorrhaging.

A microwave hematoma detection device is described in U.S. Pat. No. 6,233,479. This patent describes a non-invasive device designed to detect and locate blood pooling and clots near the outer surface of a person's body. The device is designed to find sub-dural and epidural hematomas, but it can be used to detect blood pooling anywhere near the surface of the body. The device can be modified to detect pneumothorax, organ hemorrhage, atherosclerotic plaque in the carotid arteries, and body tissue damage. It can also be used to evaluate blood flow at or near the body surface and in a number of non-destructive evaluation applications The device includes low power pulsed microwave technology, a specialized antenna, signal processing and recognition algorithms, and a disposable cap that is to be worn by a patient. The device described in the '479 patent does not detect internal hemorrhaging based on the measured physiological conditions of a person or the severity of that hemorrhaging.

A wireless medical diagnosis and monitoring device is described in U.S. Pat. No. 6,577,893. The device includes wireless electrodes, which are designed to be attached to the surface of the skin of a patient and include a digital transmitting and receiving unit, an antenna, and micro sensors. The electrodes can be used to detect EEG and EKG signals, as well as to monitor body/breathing movements, temperature, perspiration, etc. The device collects physiological data and wirelessly transmits it to a computer. The patent does not indicate that the physiological data is analyzed to determine if a person has internal bleeding or the severity of that bleeding.

U.S. Pat. No. 6,687,685 describes a system that can be used by a person to perform automated medical triage. The system generates a series of medical questions for a person, allows the person to input answers to these questions, and, when sufficient information is obtained, provides the person with a recommendation regarding obtaining further medical attention. The system uses a Bayesian Network to model medical conditions and determine the person's medical condition based on the person's responses to the series of medical questions. The '685 patent describes a general model for helping to diagnosis a disease based on medical exams and test. It does not, however, describe a system for determining if a person has internal hemorrhaging based on the person's measured physiological conditions or the severity of that hemorrhaging.

Medical intervention indicator methods and system are described in U.S. Pre-Grant Publication No. 2007/0112275. The system improves the chances of survival of an individual who has received a trauma, for example hemorrhage or blunt injury, by providing information regarding the individual to first responders including at least one of heart rate variability index value, a baroreflex sensitivity value, and a pulse pressure. This information is used in at least one implementation to provide medical treatment to injured individuals including dispatching assistance and/or prioritizing in a triage situation, increasing the speed at which these decisions can be made. In one exemplary embodiment, the heart rate variability index value is determined based on the relative power of the high frequencies versus the relative power of the low frequencies. However, the system of U.S. Pre-Grant Publication No. 2007/0112275 only uses a limited amount of information regarding the patient, and does not use a probabilistic network to make a determination of whether the patient is hemorrhaging or the severity of the hemorrhage.

What is needed, then, is system and method for detecting internal hemorrhaging in a person based on the person's measured physiological conditions and determining the severity of that hemorrhaging.

### BRIEF SUMMARY OF THE INVENTION

Methods and systems for detecting internal hemorrhaging in a person based on the person's measured physiological conditions and determining the severity of that hemorrhaging are provided. In that regard, an embodiment of a method includes the steps of measuring a plurality of physiological conditions associated with the person to generate a plurality of physiological measurements and processing these measurements using a real-time probabilistic network to determine if the person has internal hemorrhaging and the severity of that hemorrhaging. Determining the severity of the hemorrhaging is important because hemorrhaging severity determines the course of action to be taken by medical personnel.

The physiological measurements include an electrocardiogram, a photoplethysmogram, an oxygen saturation measurement, a respiratory measurement, a skin temperature measurement, and a blood pressure measurement. The step of determining internal hemorrhaging severity includes the step of determining how much blood has been lost by the person. The real-time probabilistic network classifies blood loss severity as non-specific, mild, moderate, or severe.

The processing step includes a pre-processing step and a feature extraction step. The pre-processing step includes the step of filtering the physiological measurements and the feature extraction step includes the step of extracting statistical, spectral, and temporal features from the filtered measurements. The step of filtering the physiological measurements includes the step of filtering using Fourier and wavelet filtering techniques. In a variation of this embodiment, the feature extraction step includes the step of extracting statistical, frequency, trend, magnitude transfer, non-linear, and physiological features from the filtered measurements and the real-time probabilistic network processes these extracted features

An embodiment of a system includes a plurality of physiological sensors for measuring physiological conditions associated with a person, a probabilistic network connected to the plurality of physiological sensor for detecting if the person has internal hemorrhaging and estimating the severity of that hemorrhaging based on the measured physiological conditions associated with the person, a physiological model connected to the plurality of sensors and the probabilistic network for modeling physiological conditions, and a display connected to the probabilistic network for outputting information regarding internal hemorrhaging and internal hemorrhaging severity. The plurality of sensors includes an ECG source, a blood pressure source, an SpO2 source, a respiration source, a temperature source, and a PPG source.

Embodiments of the system may provide real-time, non-invasive monitoring and detection of internal hemorrhaging in a person based on physiological measurements from the person and can be used to detect hypovolemic shock. These embodiments may be used by doctors, nurses, medics, and first responders to automatically detect internal hemorrhaging prior to availability of subjective, visible symptoms, such as degree of hypotension and nonspecific signs and subjective symptoms such as cold clammy skin, weak pulse, sweating, unstable vital signs and diminished mentation, thereby increasing the patient's chances of survival.

### BRIEF DESCRIPTION OF THE SEVERAL VIEWS OF THE DRAWINGS

Many aspects of the disclosure can be better understood with reference to the following drawings. The components in the drawings are not necessarily to scale. Moreover, in the drawings, like reference numerals designate corresponding parts throughout the several views.

FIG. 1 is a block diagram showing an embodiment of the non-invasive, early stage, hemorrhage detection system.

FIG. 2 is an exemplary plot showing four levels of internal hemorrhaging severity.

FIG. 3 is a flowchart of an embodiment of a method for processing the ECG signal.

FIG. 3 is a graphical illustration showing one embodiment of a probabilistic network.

FIG. 4 shows an example graph of an RR interval in the time domain.

FIG. 5: shows an example of the relative power spectral densities for different frequency ranges.

FIG. 6: shows an example of the trends of the relative power spectral density over time.

FIG. 7: is a flowchart Illustrating an embodiment of a method for processing blood pressure information.

FIG. 8: shows example plots of systolic, diastolic, and mean arterial blood pressure.

FIG. 9: is a flowchart Illustrating an embodiment of a method for calculating a transfer function.

FIG. 10: shows example plots of a relative transfer function.

FIG. 11: shows example trends of a relative transfer magnitude.

FIG. 12: is a flowchart of an embodiment of PPG waveform morphology calculations.

FIG. 13: shows an example of a PPG waveform.

FIG. 14: shows example morphological features of a PPG waveform.

FIG. 15: shows an example of a pulse transit time (PTT) parameter from ECG and PPG signals.

FIG. 16: shows an example plot of heart beat vs. pulse width.

FIG. 17: shows an example plot of heart beat vs. pulse transit time.

FIG. 18: is a flowchart of an embodiment of a probabilistic decision support algorithm.

FIG. 19: shows the primary components of an embodiment of a probabilistic decision support algorithm.

FIG. 20: shows an example of a probabilistic decision support algorithm.

FIG. 21: shows the components of an embodiment of a medical personnel computation node.

FIG. 22: shows the components of an embodiment of a trends computation node.

FIG. 23: shows the components of an embodiment of a physiological model node.

FIG. 24: shows the components of an embodiment of a spectral computation node.

FIG. 25: shows the components of an embodiment of a statistical computation node.

FIG. 26: shows the components of an embodiment of a transfer function computation node.

FIG. 27: shows an example of an embodiment of receiver operating curve.

### DETAILED DESCRIPTION

As discussed above, the onset of hypovolemic shock in a person is difficult to detect because the human body includes a compensatory mechanism that buffers against the noticeable changes in a person's vital signs in the early stage of shock. This is due, in part, to the fact that a small decrease of circulatory volume in the presence of adequate regulatory response reduces cardiac output without significant alterations of arterial blood pressure (ABP).

The present invention is directed toward methods and systems for non-invasive monitoring of hemorrhage that include the use of multivariate autoregressive techniques to evaluate the beat-to-beat interactions between respiration, RR interval (the time interval between two successive R waves on the ECG), and ABP. With reductions of central volume below control, baroreflex and respiratory sinus arrhythmia gains are reduced. Multivariate techniques can quantify the relations between a variety of respiratory and hemodynamic parameters, allowing for the assessment of central volume changes. Changes in pulse pressure (systolic minus diastolic), rather than arterial pressure, are useful in tracking reductions of central blood volume. In addition, changes in heart rate variability and the sensitivity of the arterial baroreflex (the ability of the heart to respond rapidly to changes in arterial pressure) also tend to change predictably as central blood volume is decreased.

The most important changes include a near-linear response of magnitude of respiratory sinus arrhythmia (RSA) and baroreflex sympathetic gain. The transfer function analysis of RSA can detect changes in autonomic response to mild degrees of central hypovolemia, which are insufficient to cause changes in mean heart rate or heart rate variance. Monitoring of pulse pressure, heart rate variability, transfer magnitude and/or baroreflex sensitivity in bleeding patients are all-important parameters in the assessment of injured patients and determination of the severity of their injury.

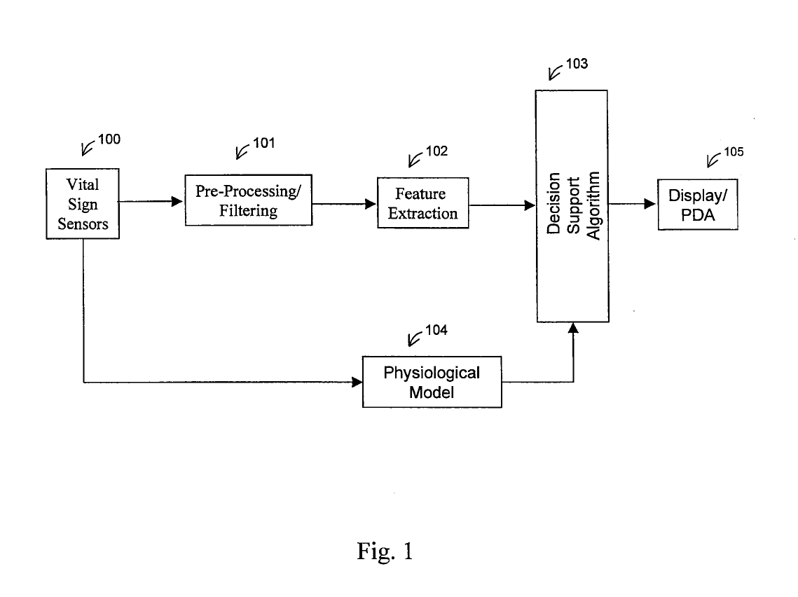

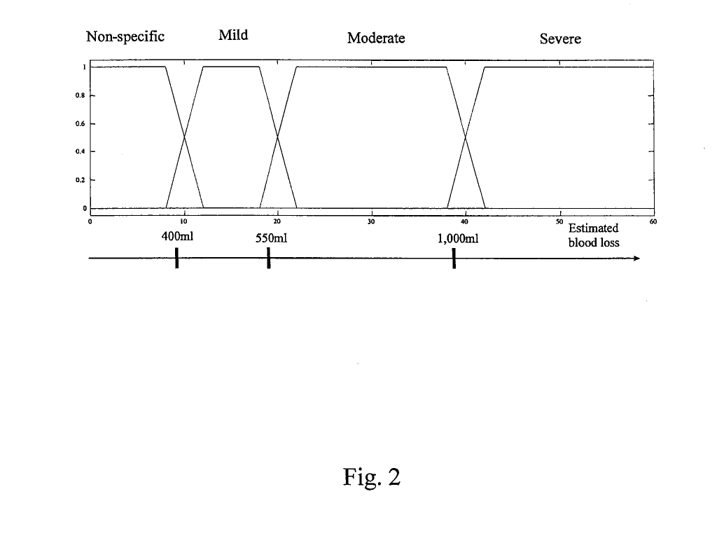

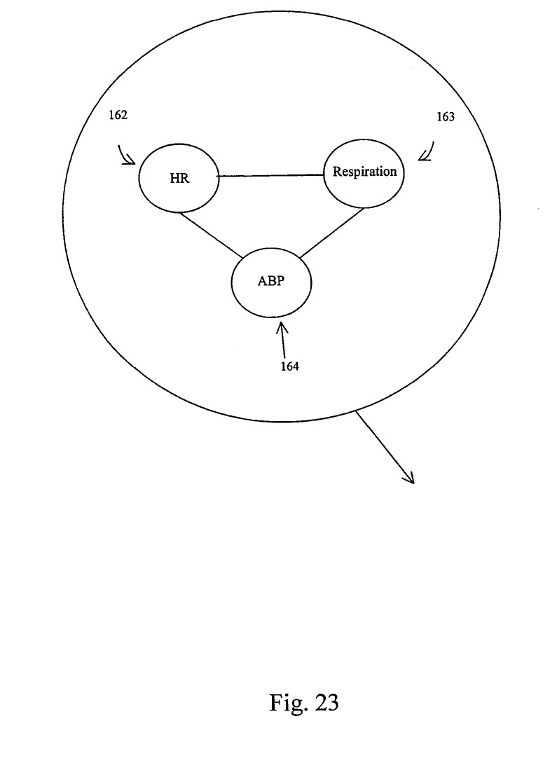

FIG. 1 is a diagram of an embodiment of a non-invasive early stage hemorrhage detection device. The input information regarding the patient is gathered from a group of vital sign sensors 100. The sensors may include an electrocardiogram (ECG), a blood pressure sensor (BP), a photoplethysmogram (PPG) waveform, an oxygen saturation sensor (SpO2), a skin temperature sensor (TEMP), and a respiratory sensor (RESP). Such sensors are well-known in the art; any appropriate sensor may be used. The information from these sensors 100 is processed by pre-processing/filtering module 101, which performs Fourier and wavelet filtering. The results of the processing are sent to feature extraction module 102, which extracts such features as statistical models, data at different frequencies, long- and short-term trends, magnitude transfer functions, and non-linear characteristics (such as fractal dimension, 1/f slope, entropy, Lyapnov exponent, principle components, and Poincare plot indices). Physiological model module 104 models the physiological conditions of the patient based on the data from the vital sign sensors; this is discussed in further detail below in the section regarding FIG. 23. The decision support algorithm 103 processes the extracted features from feature extraction module 102 and the data from the physiological model 104 using a real-time probabilistic network and assesses whether the patient is hemorrhaging, and if so, the severity of the hemorrhage. The results of the decision support algorithm are output to display 105; these results include if the patient is hemorrhaging, and if so, the severity of the hemorrhage. FIG. 2 shows a graph of the four stages that may be used to define the severity of the injury: non-specific, mild, moderate, and severe. These stages are based on the amount of blood loss.

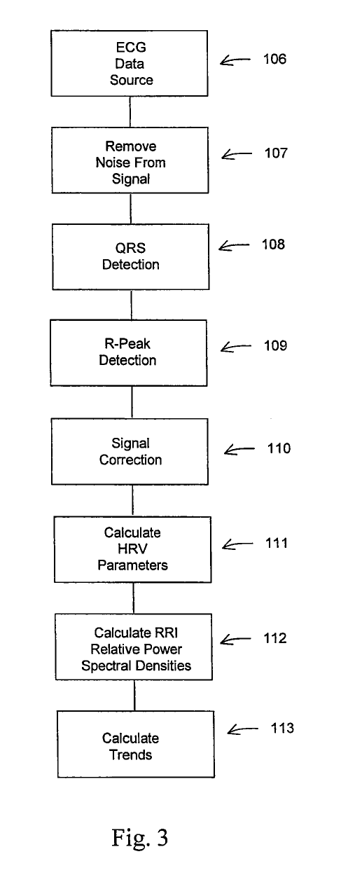

An embodiment of a pre-processing and extraction method for the ECG signal is shown in FIG. 3. The first step in processing the ECG data source 106 is to remove noise using a wavelet filter in block 107. The Continuous Wavelet Transform (CWT) of a signal x(t) is defined as

CWT(a,b)=∫x(t)ψ_{a,b}*(t)dt

where * denotes the complex conjugate, a is defined as the dilation (scale) and b is the translation (time). The basis function ψ_{a,b}(t) is obtained by scaling the prototype or mother wavelet ψ(t) at time b and scale a as follows

$${ψ}_{a , b} ( t ) = \frac{1}{\sqrt{a}} ψ ( \frac{t - b}{a} )$$

where the term 1/\sqrt{a} is introduced in order to guarantee energy preservation.

The scale may be varied to evaluate certain characteristics of a signal. As the scale parameter becomes large, the basis function becomes a stretched version of the prototype, useful for the analysis of low frequency components of the signal. In contrast, as the scale parameter becomes small, the basis function will be contracted, useful for analyzing high frequency components of the signal and detecting transients.

The CWT is a redundant representation of the signal x(t). Due to this redundancy, the CWT can be completely characterized by sampling or discretizing the parameters a and b. The most common method used to sample both on a “dyadic” grid in the time-scale plane that is a=2^{j} and b=k2^{j} which leads to

d=CWT (2^{j}, k2²)=∫x(t)ψ_{j,k}*(t)dt

where

ψ_{j,k}(t)=2^{-j/2}ψ(2^{-j}t−k)

The wavelet transform will hierarchically decompose the input signal into a series of successively lower resolution approximation signals and their associated detail signals. At each level, the approximation and detailed signals contain the information needed for reconstruction back to the next higher resolution level. One-dimensional Discrete Wavelet Transform, or 1-D DWT, processing can be described in terms of a filter bank, where an input signal is analyzed in both low and high frequency bands.

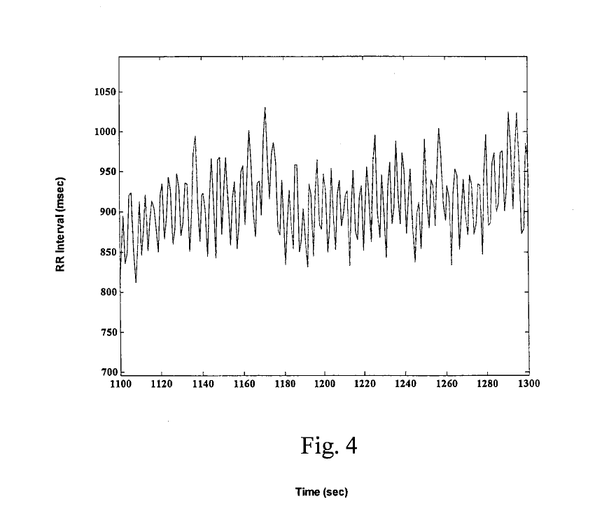

Still referring to FIG. 3, after the noise is removed from the ECG signal by the wavelet filter in block 107, the heart rate analysis proceeds by identifying the fiducial point of the ECG signal, as well as the other defined points (for example, the R point, Q point, and S point) on the ECG signal in block 108. The fiducial point is the beginning point of movement of the heart that constitutes a heartbeat, corresponding to the start of atrial depolarization and is referred to as the P point. Atrial depolarization begins in the sinoatrial (SA) node which is controlled by the autonomic nervous system. The R peak, corresponding to the point of maximum ventricular depolarization, is detected in block 109. An example R peak signal can be represented by a waveform, as shown in FIG. 4.

To identify the R peaks in block 109, the ECG signal is first filtered using a band-pass filter to reduce noise that could distort the wave. The R peaks are then identified using a differentiation and threshold algorithm to produce a pulse train, from which it is possible to identify when the derivative exceeds a set threshold. Once the R peaks are identified, the time interval between the peaks can be computed by using the pulse train to start and reset a clock. The result is a sequence of R-R durations known as the RR interval tachogram.

The next step in processing the ECG signal is to identify any ectopics or missing beats in FIG. 3, block 110. The ectopics are removed for time domain analysis. A correction process is needed in order to perform accurate frequency domain analysis. Electrical activity in the heart can affect heart rate variability analysis by causing abnormal heart beat interval wave formation. It is important not to confuse these disturbances with the modulation signal from the brain to the SA node. Thus, these erroneous signals need to be removed before performing the spectral analysis on the RR interval tachogram or instantaneous heart rate waveform. Using interpolation, these disturbances or ectopics are removed to provide the corrected heart rate signal. From this corrected heart rate signal, the interval between normal heartbeats, or the Normal-to-Normal interval (NN interval) may be determined.

Heart rate variability (HRV) parameters are calculated in block 111. This refers to the beat-to-beat alterations in heart rate. Under resting conditions, the ECG of healthy individuals exhibits periodic variation in RR intervals. This rhythmic phenomenon, known as respiratory sinus arrhythmia (RSA), fluctuates with the phase of respiration: cardio-acceleration during inspiration, and cardio-deceleration during expiration. RSA is predominantly mediated by respiratory gating of parasymphathetic efferent activity to the heart. Vagal efferent traffic to the sinus node occurs primarily in phase with expiration and is absent or attenuated during inspiration. Atropine may abolish RSA. The HRV parameters are defined as follows:

SDNN: standard deviation of all NN intervals

SDAN: standard deviation of the averages of NN intervals in all 5 min segments of the entire recording

RMSSD: the square root of the mean of the sum of the squares of differences between adjacent NN intervals.

SDNN index: mean of the standard deviations of all NN intervals for all 5 min segments of the entire recording.

SDSD: standard deviation of differences between adjacent NN intervals.

NN50 count: number of pairs of adjacent NN intervals differing by more than 50 ms in the entire recording. Three variants are possible counting all such NN intervals pairs or only pairs in which the first or the second interval is longer.

pNN50: NN50 count divided by the total number of all NN intervals.

HRV triangular index: total number of all NN intervals divided by the height of the histogram of all NN intervals measured on a discrete scale with bins.

TINN: baseline width of the minimum square difference triangular interpolation of the highest peak of the histogram of all NN intervals.

Differential index: difference between the widths of the histogram of differences between adjacent NN intervals measured at selected heights.

Logarithmic index: coefficient φ of the negative exponential curve k·e^{-φt} which is the best approximation of the histogram of absolute differences between adjacent NN intervals.

Frequency-domain analysis is a type of spectral analysis typically performed using mathematical modeling methods such as Fast Fourier Transforms (FFT) or autoregressive (AR) techniques. These techniques are used to study the frequency content of the instantaneous heart rate. In applying these techniques, a data sample is obtained over a five minute period for short term studies. FFT and AR techniques can be used to process the data sample to separate the slow responding sympathetic activities from the quicker responding parasympathetic activities. However, because these frequency domain techniques do not provide for a means to locate the time events occurring within a data sample, they are most useful for studying short term steady state conditions—situations where the data is consistent across the sample time.

In order to compensate for this shortcoming in pure frequency domain analysis, techniques have been used to modify the FFT and AR techniques to approximate a time domain analysis in addition to a frequency domain analysis. A short term FFT can be performed on smaller blocks of data from within the data sample, as opposed to using the entire data sample. This technique assumes that the data is quasi-stationary, and uses a sliding window within the data sample for choosing the data to analyze. This introduces a time dependent factor or time dependent localization into the analysis. However, this technique results in a trade-off between frequency domain analysis and time domain analysis. Choosing shorter windows within the data results in poorer frequency resolution, while increasing the window length decreases the time domain resolution. This shortcoming can create inaccuracies in the analysis of many types of biological data.

The frequency analyses of the RR intervals are calculated in FIG. 3, block 112 using standard frequency analysis techniques for each of the physiological signals. The most common is power spectral analysis. Power spectral analysis is a technique that divides the total variance in a measurement into its frequency components. In contrast, the total power obtained by integrating the power spectrum over its frequency range is equal to the total variance of the signal. The total can be calculated for specific frequency band-pass regions (in contrast to the entire spectrum). Frequency features are also extracted from the HRV signal for various power ranges. These features include:

Total power: the variance of NN intervals over the temporal segment

VLF: power in very low frequency range

LF: power in low frequency range

LF norm: LF power in normalized units

HF: Power in high frequency range

HF norm: HF power in normalized units

LF/HF: Ratio Low Frequency/High Frequency

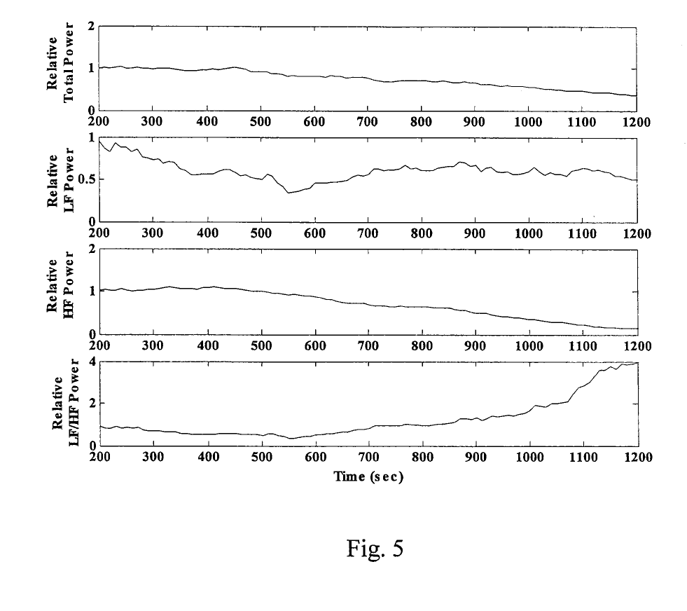

The relative values of these parameters are normalized by a maximum value, which is determined within the first 5 minutes of data collection. The relative values are important since the absolute values of all physiological measurements will vary from person to person. Example relative power spectral densities are shown in FIG. 5.

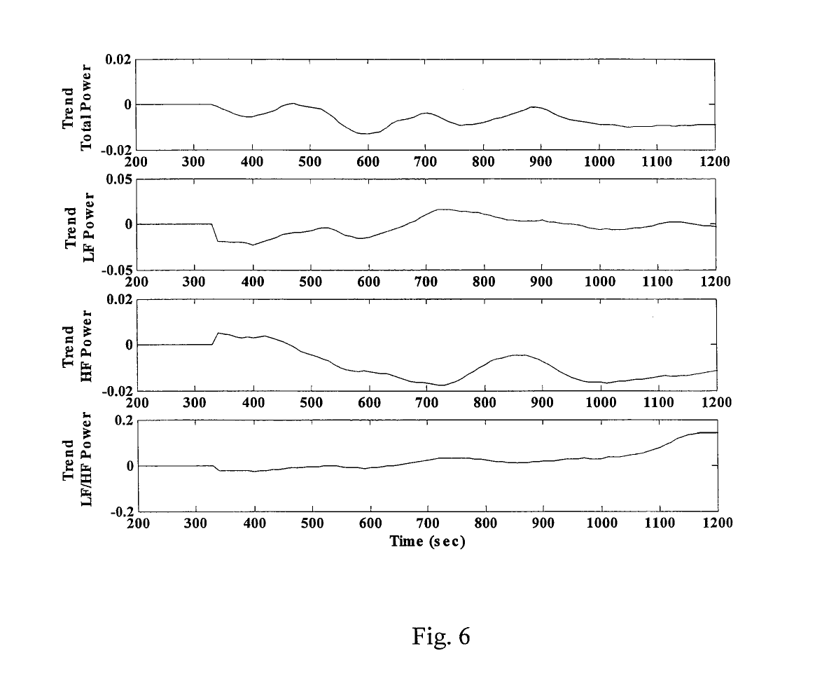

The trend analysis calculates, in FIG. 3, block 113, the change in various features/parameters over a period of time. The period can range from several minutes to several hours. Example plots of trends of the power spectral densities are shown in FIG. 6. The trend analysis can be applied to any parameter which varies with time.

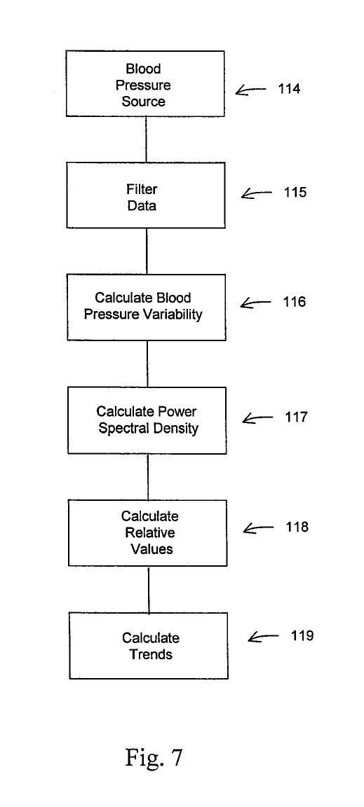

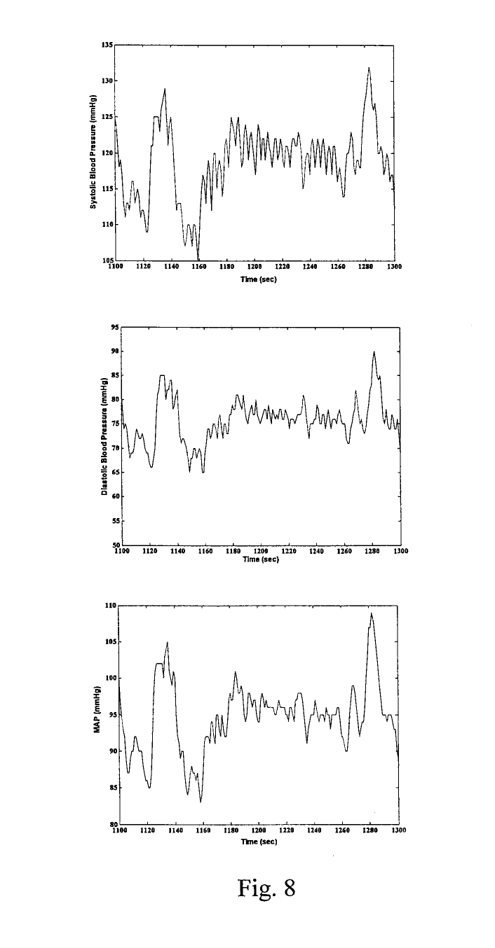

An embodiment of a pre-processing, filtering, and extraction method for blood pressure data is shown in FIG. 7. The blood pressure source monitors the patient's blood pressure in block 114 using a non-invasive blood pressure measuring method, such as the oscillometric method for burst assessment, or the Finapres method for continuous assessment. The data is passed through a low-pass Butterworth filter. A preferred embodiment uses the Finapres method, which provides the data required to perform a blood pressure variability analysis in block 116, power spectral density in block 117, relative values in block 118, and trends in block 119 in same manner as the heart rate variability calculations, as discussed above in regards to FIG. 3. Example plots of the diastolic, systolic and mean arterial pressure are shown in FIG. 8.

The transfer function of two signals defines their gain and phase relations at any given frequency and provides a statistical measure of reliability (coherence) of the relation between two signals. Evaluating transfer functions is an effective technique for investigating the relationship between the different physiological measurements. A technique that may be utilized for calculating the transfer function is based on the cross-spectral technique, given by:

$$H ( f ) = \frac{{S}_{xy} ( f )}{{S}_{xx} ( f )}$$

where H(f) represents the complex transfer function and Sₓₓ and S_{xy} represent the auto power spectrum and the cross-spectrum of the input and output signals, x and y. The cross-spectral and autospectral estimates may be computed using the Blackman-Tukey method. The transfer magnitude gain is given by:

|H(f)|={[H_{R}(f)]²+[H_{I}(f)]²}^{1/2}

where H_{R}(f) and H_{I}(f) are the real and imaginary portions of the transfer function. The transfer phase is given by:

$$Θ ( f ) = {tan}^{- 1} \frac{{H}_{I} ( f )}{{H}_{R} ( f )}$$

and the coherence is given by:

$${Coh}^{2} ( f ) = \frac{{\vert {S}_{xy} ( f ) \vert}^{2}}{{S}_{xx} ( f ) {S}_{yy} ( f )} .$$

When the transfer magnitude is defined over a specific frequency band, it is called the band-average transfer magnitude (BATM) estimate, which is given by:

$$\vert H ( band ) \vert = \frac{{\sum }_{i = 1}^{N} [ \vert {H}_{i} ( f ) \vert / {π}_{magi}^{2} ( f ) ]}{{\sum }_{i = 1}^{N} [ 1 / {π}_{magi}^{2} ( f ) ]}$$

and the i^{th} individual transfer magnitude estimate is given by:

$${π}_{magi}^{2} ( f ) = K {\vert {H}_{i} ( f ) \vert}^{2} [ \frac{1 - {Coh}_{i}^{2} ( f )}{{Coh}_{i}^{2} ( f )} ]$$

where K is a constant related to the degree of spectral smoothing.

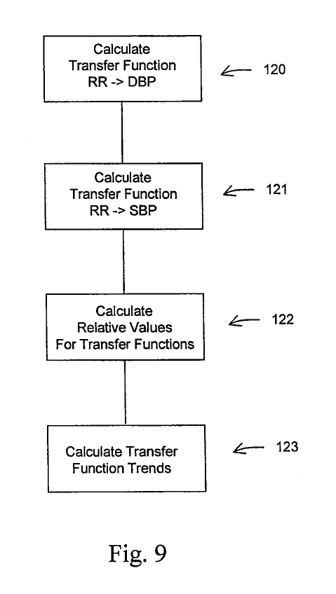

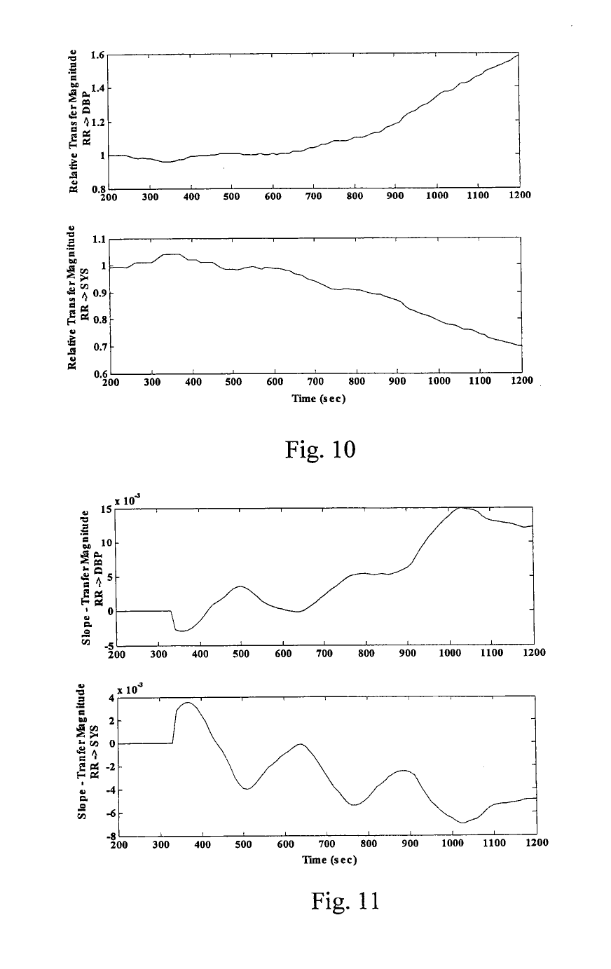

The steps for the calculating the relative transfer function parameters of the physiological data are shown in FIG. 9. The transfer function is first calculated between the RR interval and the diastolic blood pressure, indicated by RR→DBP in block 120. This is followed by the calculation of the RR interval and the systolic blood pressure, indicated by RR→SBP in block 121. The relative transfer function is calculated in block 122; an example of a plot of a magnitude of example relative transfer functions is shown in FIG. 10. The trends of the transfer magnitude of the plots of FIG. 10 are shown in FIG. 11. The transfer function phase, magnitude, and coherence may all be calculated.

Photoplethysmography (PPG) relates to the use of optical signals transmitted through or reflected by a patient's blood, e.g., arterial blood or perfused tissue, for monitoring a physiological parameter of a patient. Such monitoring is possible because the optical signal is modulated by interaction with the patient's blood. That is, interaction with the patient's blood generally involves a wavelength and/or time dependent attenuation due to absorption, reflection and/or diffusion, and imparts characteristics to the transmitted signal that can be analyzed to yield information regarding the physiological parameter of interest. Such monitoring of patients is desirable because it is noninvasive, typically yields substantially instantaneous and accurate results, and utilizes minimal medical resources, thereby proving to be cost effective.

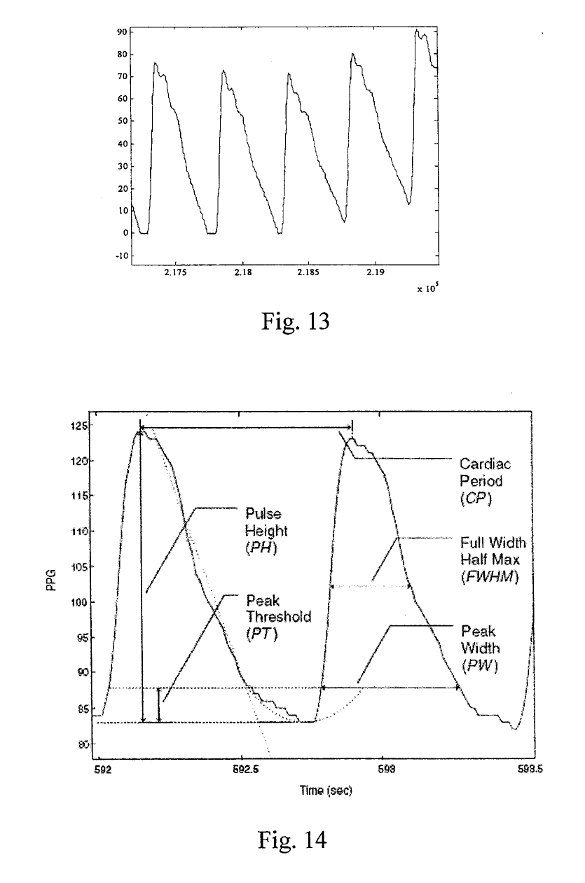

A common type of photoplethysmographic (PPG) instrument is the pulse oximeter. Pulse oximeters determine an oxygen saturation level (Spo2) of a patient's blood, or related analyte values, based on transmission/absorption characteristics of light transmitted through or reflected from the patient's tissue. In particular, pulse oximeters generally include a probe for attaching to a patient's appendage such as a finger, earlobe or nasal septum. The probe is used to transmit pulsed optical signals of at least two wavelengths, typically red and infrared, through the patient's appendage. The transmitted signals are received by a detector that provides an analog electrical output signal representative of the received optical signals. By processing the electrical signal and analyzing signal values for each of the wavelengths at different portions of a patient's pulse cycle, information can be obtained regarding blood oxygen saturation. In addition, temporal variation in blood volume of peripheral tissue, and thus blood flood, can be measured noninvasively using an optically-based pulse oximeter. The changes in light absorption caused by the volumetric change in blood in the tissue beneath the sensor gives a photometric based plethysmogram. An example of a PPG waveform is shown in FIG. 13.

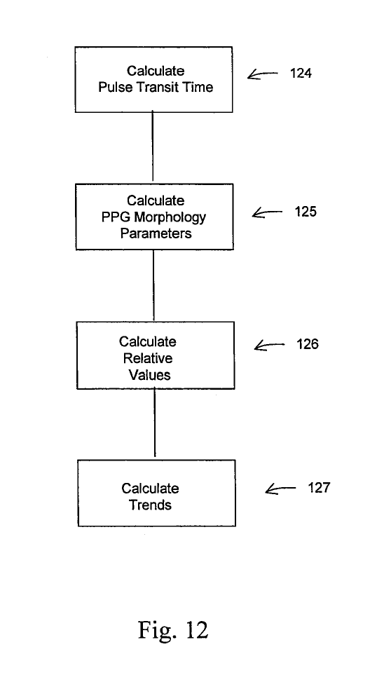

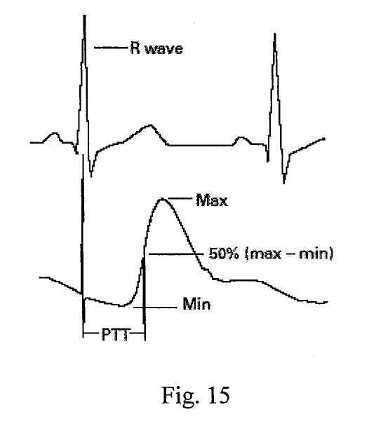

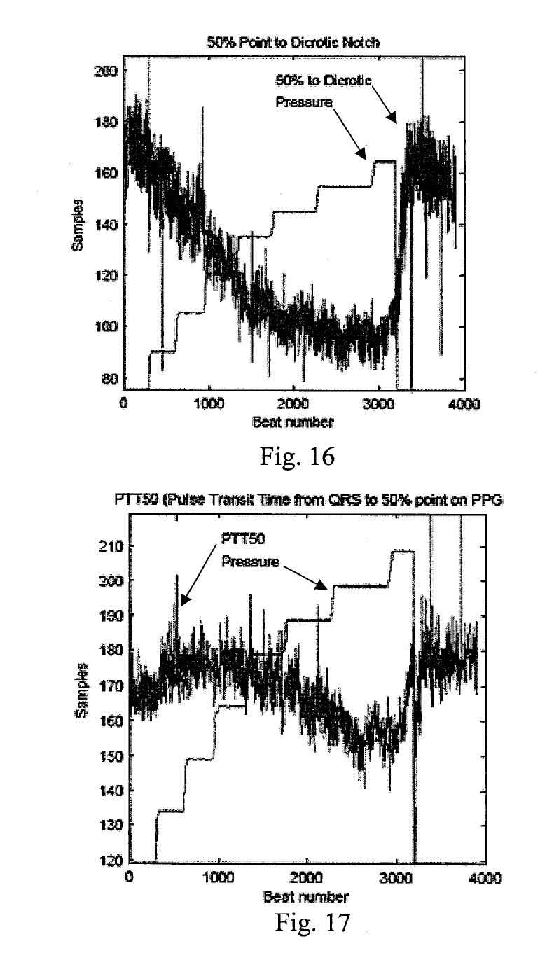

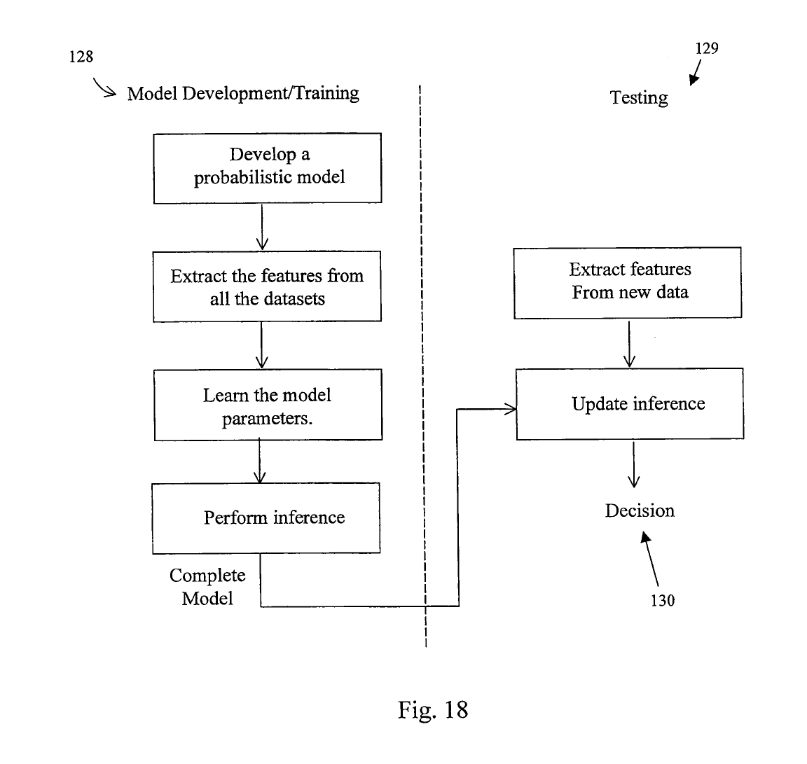

An embodiment of a pre-processing and extraction method for PPG and SpO2 calculations is shown in FIG. 12. In block 124, the Pulse Transit Time (PTT) is calculated. PTT can be defined as the interval between ventricular electrical activity and the appearance of a peripheral pulse waveform, as shown in FIG. 15. PTT can encompasses three timing elements: the time from the onset of ventricular electrical activity to the beginning of ejection into the aorta or the cardiac pre-ejection period (PEP), or the electromechanical delay; the interval from aortic pulse emergence to the arrival of its initial upstroke at the monitoring site, or arterial transit time; and the duration measured from the start of the arterial pulse waveform upstroke to the point at which pulse arrival is detected, or rise time of the pulse. A graphical representation of the pulse width of a patient with simulated internal bleeding is shown in FIG. 16. A graphical representation of the pulse transit time of a patient with simulated internal bleeding is shown in FIG. 17.

Still referring to FIG. 12, in block 125, the PPG morphology parameters are calculated. The morphology features used to characterize an example pulse are shown in FIG. 14. The Pulse Height (PH) is the difference between the maximum of a cardiac cycle and the previous minimum. The Cardiac Period (CP) is the difference in time between the peaks of two consecutive cardiac cycles. The Full Width Half Max (FWHM) is the width of the peak at half the maximum value of the cardiac cycle. The Peak Width (PW) is the width of the peak at a predetermined Peak Threshold (PT). The Normalized Peak Width (NPW) is the PW divided by the Cardiac Period (CP). A key feature in the detection of hemorrhaging is the pulse width.

The relative values of the PPG morphology parameters are calculated in FIG. 12, block 126, by dividing each parameter by its maximum value. The trend parameters are determined in FIG. 12, block 127 by calculating the slope of each parameter over, for example, a five minute window for short-term trends and 30 minute window for long-term trends.

Temperature and respiratory signal processing includes the use of standard Fourier filters to remove unwanted noise. The maximum and minimum of each complete cycle of the respiratory signal are also extracted from the respiratory signal.

FIG. 18 shows a method of an embodiment of a decision support algorithm, which performs a decision assessment based both on the extracted features of the information that was gathered from the sensors and the information from the physiological model. The decision support algorithm evaluates whether the patient is hemorrhaging and, if so, the severity of the hemorrhage. The decision support algorithm is based on a probabilistic decision network that is a compact representation of a joint probability distribution on a problem domain. A probabilistic network models qualitative and quantitative knowledge about the problem domain, in this case, the physiological model data and the extracted features of the processed data from the vital sign sensors that are fed into it.

The development of probabilistic networks is based on Bayes Rule, which relates the conditional and marginal probability distributions of random variables. In some interpretations of probability, Bayes' theorem may be used to update or revise beliefs in light of new evidence a posteriori.

The probability of an event A conditional on another event B is generally different from the probability of B conditional on A. However, there is a definite relationship between the two, and Bayes' theorem states that relationship. The conditional relationship is given by:

$$P ( A \vert B ) = \frac{P ( B \vert A ) P ( A )}{P ( B )}$$

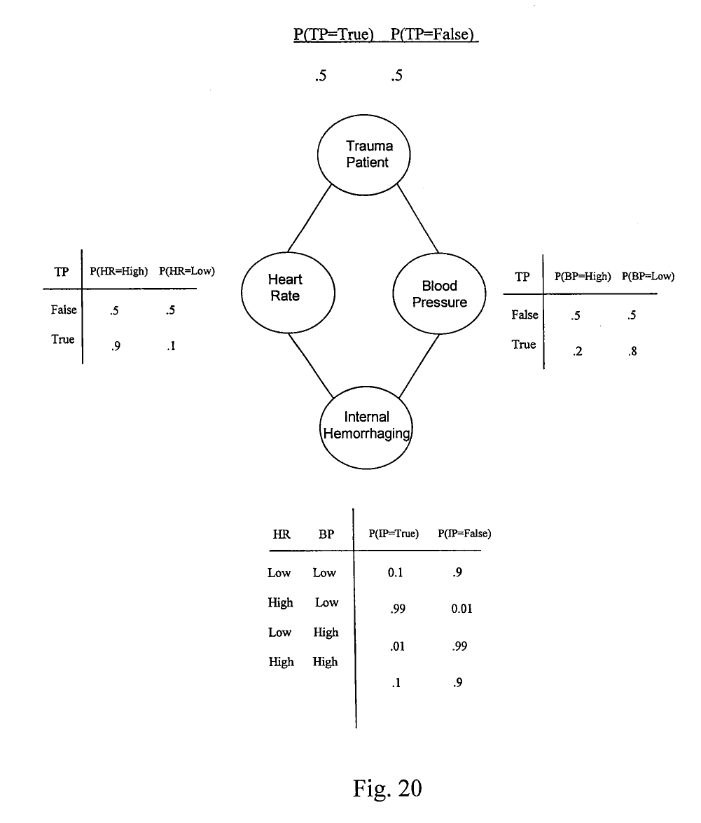

FIG. 20 shows a model of the probabilistic relationships between discrete physiological variables. The model is defined by Conditional Probability Distributions (CPD). Each of the variables may be represented by Conditional Probability Table (CPT) which defines the probability that the child node takes on each of its different values for each combination of values of its parents. To mathematically define the probabilistic relationships between each of the nodes of the model, the chain rule of probability is used to define the joint probability of all the nodes in the model. For this model, the joint probability is given by

P(TP,HR,BP,TH)=P(TP)*P(HR|TP)*PBP|TP,HR)*P(IH|TP,HR,BP)

The nodes are defined as:
TP: Trauma Patient
HR: Heart Rate
BP: Blood Pressure
IH: Internal Hemorrhaging

By using conditional independence relationships, this can be rewritten as

P(TP,HR,BP,IH)=P(TP)*P(HR|TP)*PBP|TP)*P(IH|HR,BP)

In this example, the event of internal hemorrhaging (IH) is determined by the heart rate (HR) and blood pressure (BP). The strength of this relationship is inferred by the joint probabilities of each of the nodes. For example, P(IH=True|HR=High, BP=Low)=0.99 and P(IH=False|HR=Low, BP=High)=0.01.

The calculations for the joint probability distributions for this model are relatively simple, but for more complex models, direct implementation of the chain rule is computationally impractical in real time. Therefore a variety of approximation techniques have been developed to fully specify all the probabilistic elements of the model. Probabilistic networks provide a method of both representing conditional independence between random variable and computing the probability distributions associated with these random variables. In a probabilistic network, a joint probability distribution is represented using a directed graph.

The probabilistic network architecture allows for the incorporation of information as it becomes available and also allows for the incorporation of expert knowledge. This knowledge can be propagated throughout the network and, as more knowledge is used, better estimates can be made. This structure allows an estimate to be made even when only partial information is available at a given state.

In order to fully specify the probabilistic network, it is necessary to further specify for each node the probability distribution for the node conditional upon the node's parents. The distribution of the node conditional upon its parents may have any form. It is common to work with discrete or Gaussian distributions since that simplifies calculations. Sometimes only the constraints on a distribution are known; the principle of maximum entropy may be used under these circumstances to determine a single distribution, which is the one with the greatest entropy given the constraints.

Often these conditional distributions include parameters which are unknown and must be estimated from data, sometimes using the maximum likelihood approach. Direct maximization of the likelihood (or of the posterior probability) is often complex when there are unobserved variables. A classical approach to this problem is the expectation-maximization algorithm which alternates computing expected values of the unobserved variables conditional on observed data, with maximizing the complete likelihood (or posterior) assuming that previously computed expected values are correct. Under mild regularity conditions this process converges on maximum likelihood (or maximum posterior) values for parameters. All these methods are described in a book entitled “Learning Bayesian Networks”, authored by R. E. Neopolitan, and published by Prentice Hall in 2003, which is hereby incorporated by reference.

Because a probabilistic network is a complete model for the variables and their relationships, it can be used to answer probabilistic queries about them. For example, the network can be used to find out updated knowledge of the state of a subset of variables as other variables (the evidence variables) are observed. This process of computing the posterior distribution of variables given ongoing evidence collection is called probabilistic inference. The posterior gives a universal sufficient statistic for detection applications, when one wants to choose values for the variable subset which minimize some expected loss function, for instance the probability of decision error. A probabilistic network can thus be considered a mechanism for automatically constructing extensions of Bayes' theorem to more complex problems.

The most common exact inference methods are variable elimination, which eliminates (by integration or summation) the non-observed non-query variables one by one by distributing the sum over the product; clique tree propagation, which caches the computation so that many variables can be queried at one time and new evidence can be propagated quickly; and recursive conditioning, which allows for a space-time tradeoff and matches the efficiency of variable elimination when enough space is used. All of these methods have complexity that is exponential to the network's tree width. The most common approximate inference algorithms are stochastic MCMC simulation, mini-bucket elimination which generalizes loopy belief propagation, and variational methods.

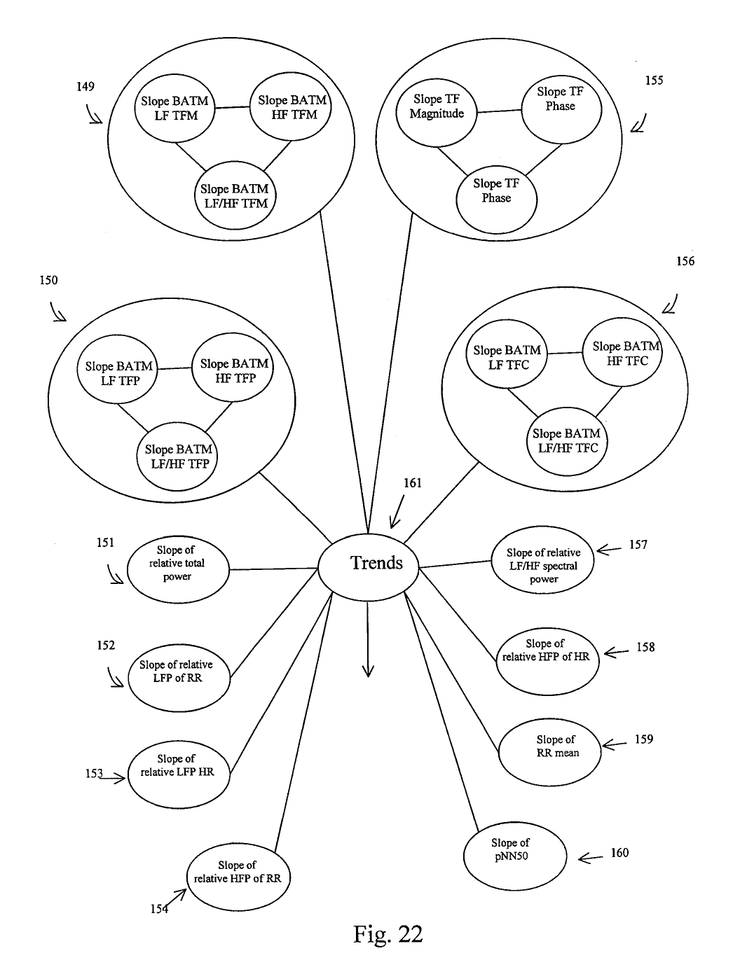

The overall steps involved in implementing the probabilistic network used in an embodiment of the decision support algorithm are shown in FIG. 18. The overall early stage hemorrhage detection model is divided into two components: a model development/training module 128 and model testing module 129. The model is developed utilizing vital sign data from a trauma patient and medical understanding of the human physiological response to trauma and hemorrhaging. The development training module 128 develops a probabilistic model using the features that have been extracted from the pre-processed and filtered physiological measurements (see discussion of FIGS. 22, 24, 25, and 26, below, for further discussion of feature extraction) and learning the parameters from the extracted features. Then inference is performed with the learned model parameters. Once the model is developed by development training module 128, it is ready for testing by module 129 with new data, which involves extracting features from the new data and performing updated inference in order to determine if the patient is hemorrhaging internally. The output is given as a probability at decision block 130.

Embodiments of the development training module 128 automatically infer a structure of the probabilistic model from a set of possible models using the current state of the patient and the corresponding values of the variables. The inferred structure of the probabilistic model inferred is the model most likely to produce the status of the patient under observation. Training cases and model variables are used to automatically learn model parameters and to compute prior and conditional probability densities of variables considered in the structured probabilistic model. Probability densities are used to automatically produce a hemorrhage detection model and injury severity model for accurately approximating the current state of the patient. The data from the various vital sign sensors is processed based on inferred probabilities to estimate the patient's status. The training module 128 is capable of automatically inferring a probabilistic dependency structure among variables in a probabilistic network model, and probability densities characterizing the dependencies. It is also capable of using probabilistic learning methods to infer hidden variables, dependencies, and probability densities of variables in a probabilistic network model.

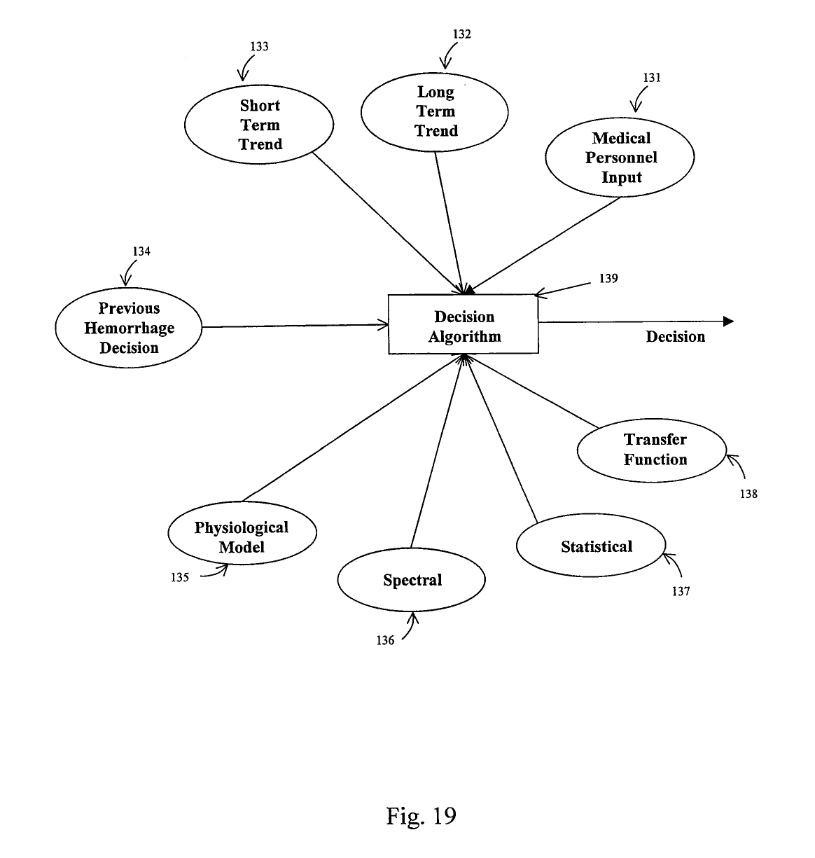

Embodiments of a real-time probabilistic network associated with an embodiment of the decision support algorithm temporally process the extracted features of the physiological measurements and physiological model information. More specifically, embodiments of the probabilistic network output a decision based on the following input information, or nodes, as shown in FIG. 19: medical personnel input 131, long term trends 132, short term trends 133, previous hemorrhage decisions 134, physiological model 135, spectral features 136, statistical features 137, and transfer function features 138. Each of these nodes has a set of input nodes comprising information from the vital sign sensors and/or the feature extraction module, and will be discussed in greater detail below. Some of these nodes, specifically, the long and short term trends, the spectral features, the statistical features, and the transfer function features, are functionally part of the feature extraction module of FIG. 1, element 102, but as they interact closely with the decision support algorithm, they are described in further detail below.

Embodiments of the real-time probabilistic network use a Bayesian network to determine if hemorrhaging is present based on the information from all the nodes, and output a decision as to whether the patient is hemorrhaging at node 139. If hemorrhaging is determined to be present, then the probabilistic network further determines the severity of the injury based on an estimation of the blood loss. The injury severity may be classified into one of four categories: non-specific, mild, moderate and severe.

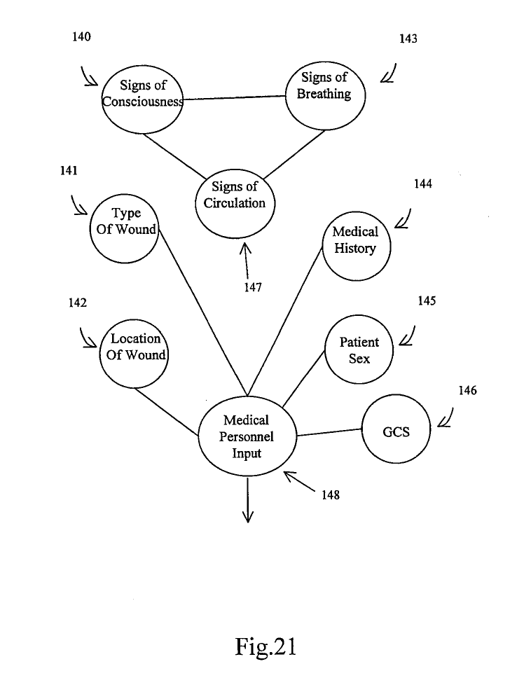

An embodiment of the medical personnel node 131 is a probabilistic network composed of seven input nodes, as shown in FIG. 21. These nodes include signs of consciousness 140, type of wound 141, location of wound 142, signs of breathing 143, medical history 144, patient gender 145, Glasgow Coma Scale (GCS) rating 146, and signs of circulation 147. Each of these nodes is qualitative in nature and are standard assessments made by emergency medical personnel. The output probability at node 148 is a probability that is based on an overall assessment of the values of all of the input nodes.

Embodiments of the short term trends 133 and long term trends 132 feature extraction nodes are shown in FIG. 22. The same model structure is used for both short and long term trend determinations, the difference being that the parameters are calculated for different time windows. Short term trends may be calculated for a window of less than 3 minutes, and long term trends may be calculated for a window greater than 3 minutes, for example. The trends probabilistic network is composed of twelve input nodes: BATM Transfer function magnitude 149, BATM transfer function phase 150, slope of relative total power 151, slope of relative low frequency power of RR 152, slope of relative low frequency power of heartrate 153, slope of relative high frequency power of RR 154, slope of transfer phase 155, BATM transfer function coherence 156, slope of relative low frequency/high frequency spectral power 157, slop of relative high frequency power of heartrate 158, slope of RR mean 159, and slope of pNN50 160. At node 161 an overall trend is determined from the input nodes and is output to the decision algorithm.

A first embodiment of a physiological model node 135 (also element 104 of FIG. 1) is shown in FIG. 23. The physiological model receives information directly from the vital signs sensors 100 of FIG. 1. It is based on a trivariate model, and will calculate the estimated heart rate 162, blood pressure 163, and respiration 164. The trivariate model is described in a paper entitled “Heart Rate Control and Mechanical Cardiopulmonary Coupling to Assess Central Volume: a Systems Analysis” and published in the American Journal of Physiology—Regulatory Integrative and Comparative Physiology, on Nov. 1, 2002; 283(5):R1210-1220, by R. Barvieri, J. K. Triedman, and J. P. Saul, which is hereby incorporated by reference. The estimates generated by the trivariate model will be compared with the actual measurements, producing an error signal.

In a second embodiment of a physiological model node 135, the physiological parameters may be computed based on a cardiovascular short-term regulation model. This is a multivariate autoregressive technique which models the beat-to-beat interactions between respiration, RR interval, central venous pressure (CPV), and arterial blood pressure (APB). Relationships between biological signals can be attributed to specific physiological mechanisms, and this multivariate technique may be used to quantify the relations between the respiratory and the hemodynamic parameters, allowing for assessment of central volume changes. The most important changes include a near-linear response of magnitude of respiratory sinus arrhythmia (RSA) and baroreflex sympathetic gain. The model output is compared with the actual measurements to output the error signal.

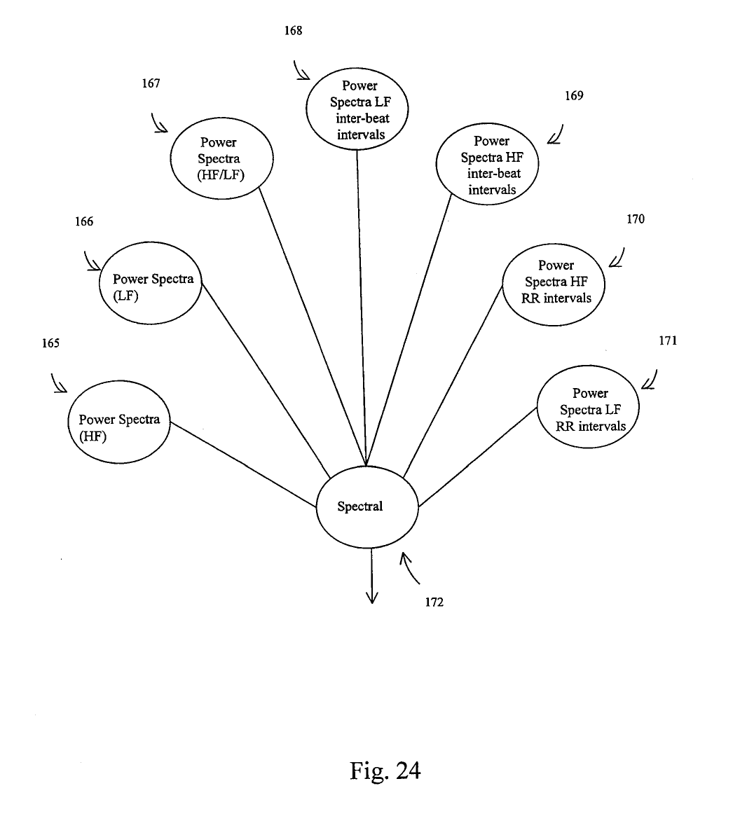

An embodiment of the spectral feature extraction node 136 is shown in FIG. 24. This node processes spectral calculations for each of the vital sign measurements. These calculations are performed utilizing standard Fourier spectral analysis techniques. This model is composed of seven input nodes: the high-frequency power spectrum (for HR, APB, DBP, PPG, SpO2, and RESP) 165, the low-frequency power spectrum (for HR, APB, DBP, PPG, SpO2, and RESP) 166, the ratio of the high and low frequency power spectra (for HR, APB, DBP, PPG, SpO2, and RESP) 167, the low frequency power spectra of the inter-beat intervals 168, the high-frequency power spectra of the inter-beat intervals 169, the high-frequency power spectra of the RR intervals 170, and the low-frequency power spectra of the RR intervals 171. The overall spectral output is given at node 172 and is then input into the decision support algorithm.

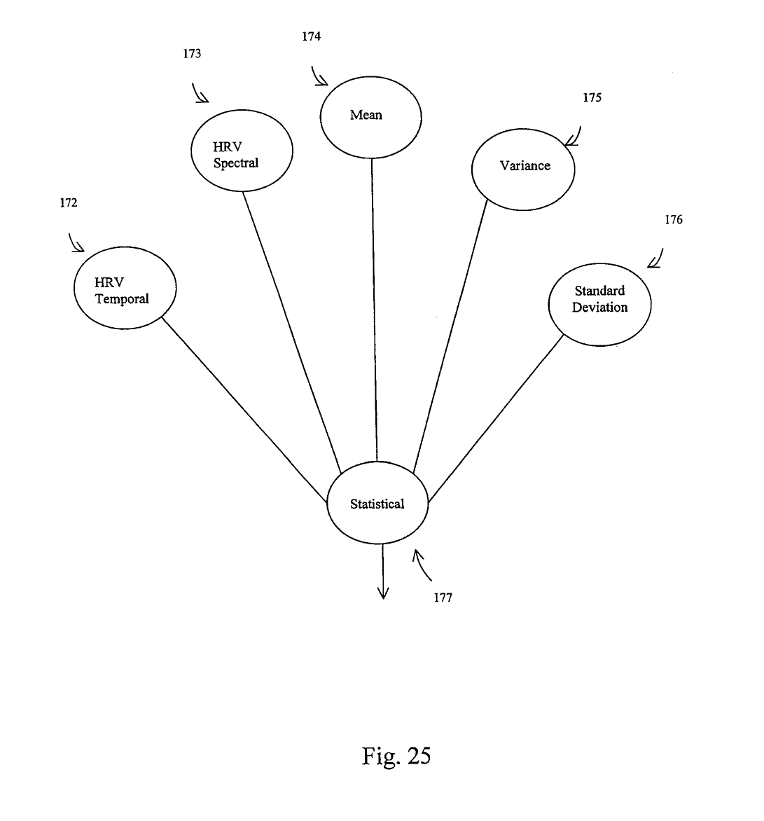

An embodiment of a statistical feature extraction node 137 is shown in FIG. 25. This model incorporates statistical calculations for each of the vital sign measurements. The spectral and temporal statistical HRV (heart rate variability) components are standard calculations utilized by the cardiology researchers, as explained in a paper published by the Task Force of the European Society of Cardiology and the North American Society of Pacing and Electrophysiology in the European Heart Journal in 1996 (vol. 17, pp. 354-381), entitled “Guidelines, Heart Rate Variability, Standards of Measurement, Physiological Interpretation, and Clinical Use”, which is hereby incorporated by reference. The other elements of this model are derived utilizing standard statistical calculations. This model is composed of five input components: the temporal heart rate variability 172, the spectral heart rate variability 173, the mean heart rate variability 174, the variance of the HRV 175, and the standard deviation of the HRV 176. The mean and variance are calculated for each input, as well as the percentage of RR interval differences larger than 50 ms. The overall statistical analysis is given in node 177.

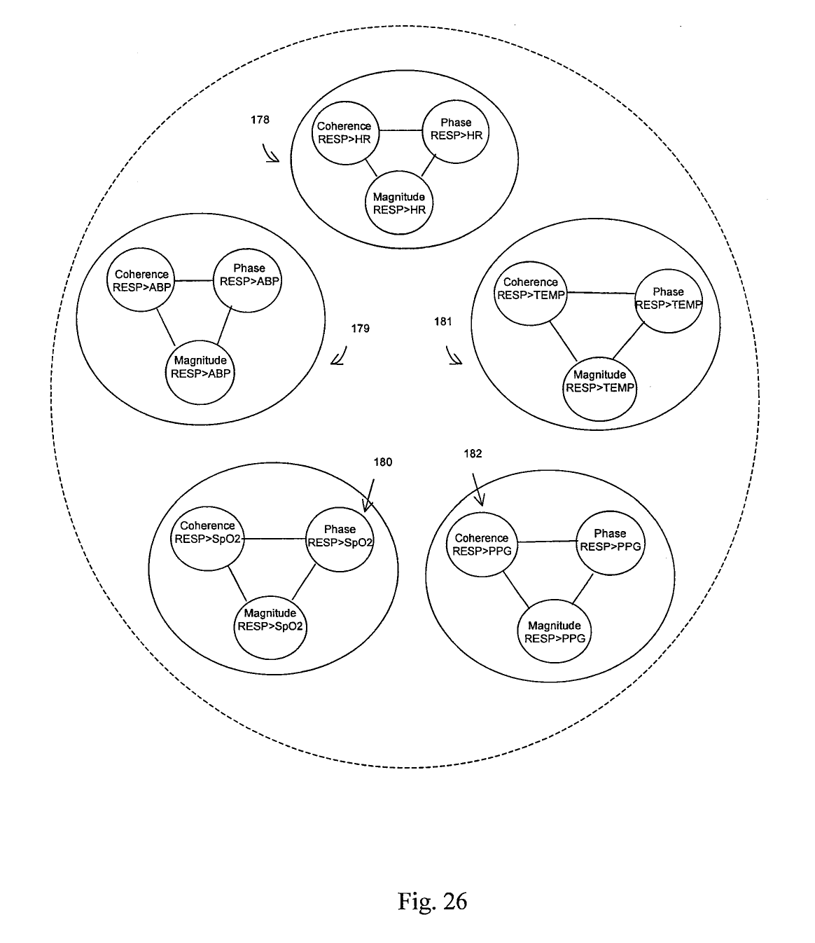

An embodiment of a transfer function feature extraction node 138 is shown in FIG. 26. This model calculates transfer functions (magnitude, phase, and coherence) for each of the vital sign measurements in terms of the other components. This model is composed of five components per sensor input, for example, for respiration: respiration>heartrate 178, respiration>APB 179, Respiration>Oxygen saturation 180, respiration>temperature 181, and respiration>photoplethysmography 182. The relative transfer functions may be similarly calculated for other sensor source combinations, i.e., APB>HR, APB>RESP, APB>SpO2, APB>PPG, APB>TEMP, SpO2>RESP, SpO2>APB, SpO2>HR, SpO2>PPG, and SpO2>TEMP. The BATM may be calculated for each sensor source combination as well, for both low and high frequencies

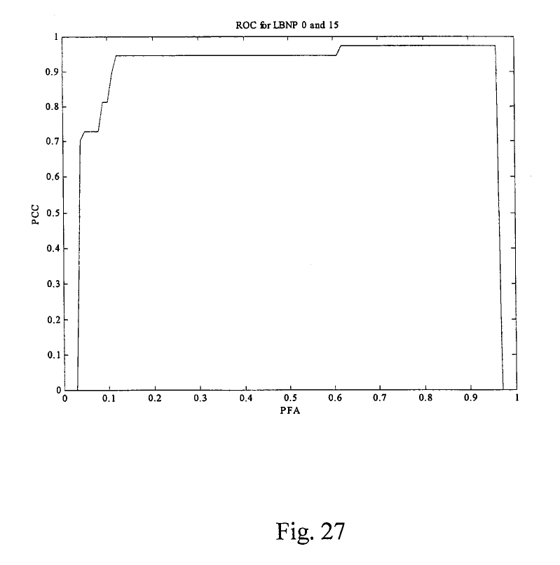

The information from each of the above-described nodes is fed into the decision support algorithm, which uses a real-time Bayesian probabilistic network, as is described above, to arrive at two overall outputs. The first is a probability that the patient in internally hemorrhaging. A receiver operator curve that may be output by the decision support algorithm is shown in FIG. 27. The second output is an estimate of the severity of the injury of the patient, which may fall into one of four categories (non-specific, mild, moderate, or severe), based on the estimated blood loss. The outputs of the network may be sent to a display device, which may be a computer display or personal digital assistant (PDA).

Various functionality, such as that described above in the flowcharts and/or the functionality described with respect to computational algorithms, can be implemented in hardware and/or software. In this regard, a computing device can be used to implement various functionality, such as the pre-processing/filtering module 101, feature extraction module 102, physiological model 104, or the decision support algorithm 103 of FIG. 1.

In terms of hardware architecture, such a computing device can include a processor, memory, and one or more input and/or output (I/O) device interface(s) that are communicatively coupled via a local interface. The local interface can include, for example but not limited to, one or more buses and/or other wired or wireless connections. The local interface may have additional elements, which are omitted for simplicity, such as controllers, buffers (caches), drivers, repeaters, and receivers to enable communications. Further, the local interface may include address, control, and/or data connections to enable appropriate communications among the aforementioned components.

The processor may be a hardware device for executing software, particularly software stored in memory. The processor can be a custom made or commercially available processor, a central processing unit (CPU), an auxiliary processor among several processors associated with the computing device, a semiconductor based microprocessor (in the form of a microchip or chip set) or generally any device for executing software instructions.

The memory can include any one or combination of volatile memory elements (e.g., random access memory (RAM, such as DRAM, SRAM, SDRAM, VRAM, etc.)) and/or nonvolatile memory elements (e.g., ROM, hard drive, tape, CD-ROM, etc.). Moreover, the memory may incorporate electronic, magnetic, optical, and/or other types of storage media. Note that the memory can also have a distributed architecture, where various components are situated remotely from one another, but can be accessed by the processor.

The software in the memory may include one or more separate programs, each of which includes an ordered listing of executable instructions for implementing logical functions. A system component embodied as software may also be construed as a source program, executable program (object code), script, or any other entity comprising a set of instructions to be performed. When constructed as a source program, the program is translated via a compiler, assembler, interpreter, or the like, which may or may not be included within the memory.

The Input/Output devices that may be coupled to system I/O Interface(s) may include input devices, for example but not limited to, a keyboard, mouse, scanner, microphone, camera, proximity device, etc. Further, the Input/Output devices may also include output devices, for example but not limited to, a printer, display, etc. Finally, the Input/Output devices may further include devices that communicate both as inputs and outputs, for instance but not limited to, a modulator/demodulator (modem; for accessing another device, system, or network), a radio frequency (RF) or other transceiver, a telephonic interface, a bridge, a router, etc.

When the computing device is in operation, the processor can be configured to execute software stored within the memory, to communicate data to and from the memory, and to generally control operations of the computing device pursuant to the software. Software in memory, in whole or in part, is read by the processor, perhaps buffered within the processor, and then executed.

One should note that the flowcharts included herein show the architecture, functionality, and operation of a possible implementation of software. In this regard, each block can be interpreted to represent a module, segment, or portion of code, which comprises one or more executable instructions for implementing the specified logical function(s). It should also be noted that in some alternative implementations, the functions noted in the blocks may occur out of the order and/or not at all. For example, two blocks shown in succession may in fact be executed substantially concurrently or the blocks may sometimes be executed in the reverse order, depending upon the functionality involved.

One should note that any of the functionality described herein can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” contains, stores, communicates, propagates and/or transports the program for use by or in connection with the instruction execution system, apparatus, or device. The computer readable medium can be, for example but not limited to, an electronic, magnetic, optical, electromagnetic, infrared, or semiconductor system, apparatus, or device. More specific examples (a nonexhaustive list) of a computer-readable medium include a portable computer diskette (magnetic), a random access memory (RAM) (electronic), a read-only memory (ROM) (electronic), an erasable programmable read-only memory (EPROM or Flash memory) (electronic), and a portable compact disc read-only memory (CDROM) (optical).

It should be emphasized that the above-described embodiments are merely possible examples of implementations set forth for a clear understanding of the principles of this disclosure. Many variations and modifications may be made to the above-described embodiments without departing substantially from the spirit and principles of the disclosure. All such modifications and variations are intended to be included herein within the scope of this disclosure and protected by the accompanying claims.

What is claimed is:

1. A method for non-invasively detecting internal hemorrhaging in a person, comprising the steps of:
measuring a plurality of physiological conditions associated with a person to generate a plurality of physiological measurements; and
processing the plurality of physiological measurements using a real-time decision algorithm to determine if the person has internal hemorrhaging and, if so, internal hemorrhaging severity.

2. The method of claim 1, wherein the plurality of physiological measurements includes an electrocardiogram, a photoplethysmogram, an oxygen saturation measurement, a respiratory measurement, a skin temperature measurement, a blood pressure measurement, and a Glasgow coma score measurement.

3. The method of claim 1, wherein the step of determining internal hemorrhaging severity includes the step of determining how much blood has been lost by the person.

4. The method of claim 3, wherein the real-time decision algorithm classifies blood loss by the person as non-specific blood loss, mild blood loss, moderate blood loss, or severe blood loss.

5. The method of claim 1, wherein:
the processing step includes a pre-processing step and a feature extraction step;
the pre-processing step includes the step of filtering the plurality of physiological measurements to generate a plurality of filtered physiological measurements; and
the feature extraction step includes the step of extracting statistical, spectral, and temporal features from the plurality of filtered physiological measurements.

6. The method of claim 5, wherein the step of filtering the plurality of physiological measurements includes the step of filtering the plurality of physiological measurements using Fourier and wavelet filtering.

7. The method of claim 1, wherein:
the processing step includes a feature extraction step;
the feature extraction step includes the step of extracting statistical features, frequency features, trend features, transfer function features, non-linear features, and physiological features; and
the real-time decision algorithm processes the statistical, frequency, trend, transfer function, non-linear, and physiological features.

8. The method of claim 1, wherein the processing step includes the step of calculating correlations between the plurality of physiological measurements.

9. A system for detecting and estimating internal hemorrhaging severity in a person, comprising:
a plurality of physiological sensors for measuring physiological conditions associated with a person; and
a real-time probabilistic network connected to the plurality of physiological sensors for detecting if the person has internal hemorrhaging and estimating internal hemorrhaging severity based on the measured physiological conditions associated with the person.

10. The system of claim 9, wherein the plurality of physiological sensors includes an electrocardiogram, a photoplethysmogram, an oxygen saturation sensor, a respiratory sensor, a skin temperature sensor, and a blood pressure sensor.

11. The system of claim 9, wherein the real-time probabilistic network determines how much blood has been lost by the person.

12. The system of claim 11, wherein the real-time probabilistic network classifies the amount of blood lost by the person as non-specific blood loss, mild blood loss, moderate blood loss, or severe blood loss.

13. The system of claim 9, wherein:
the real-time probabilistic network performs pre-processing, the pre-processing comprising filtering the plurality of physiological measurements to generate a plurality of filtered physiological measurements, and feature extraction, the feature extraction comprising extracting statistical, spectral, and temporal features from the plurality of filtered physiological measurements, of the measured physiological conditions.

14. The system of claim 13, wherein filtering the plurality of physiological measurements includes filtering the plurality of physiological measurements using Fourier and wavelet filtering.

15. The system of claim 9, wherein:
the real-time probabilistic network performs feature extraction, the feature extraction comprising extracting statistical features, frequency features, trend features, transfer function features, non-linear features, and physiological features; and
the real-time probabilistic network processes the extracted statistical, frequency, trend, transfer function, non-linear, and physiological features.

16. A system for detecting and estimating internal hemorrhaging severity in a person, comprising:
a plurality of vital sign sensors operative to take physiological measurements from the person;
a physiological modeling module, operative to receive the physiological measurements from the plurality of vital sign sensors;
a pre-processing module and filtering module, operative to receive the physiological measurements from the plurality of vital sign sensors;
a feature extraction module, operative to receive the filtered physiological measurements from the pre-processing and filtering module; and
a decision support algorithm, the decision support algorithm comprising a real-time probabilistic network for detecting and estimating the severity of internal hemorrhaging in the person based on the outputs of the physiological modeling module, the pre-processing and filtering module, and the feature extraction module.

17. The system of claim 16, wherein the plurality of vital sign sensors comprise: an electrocardiogram, a blood pressure sensor, a photoplethysmogram, an oxygen saturation sensor, a respiratory sensor, and a skin temperature sensor.

18. The system of claim 16, wherein the physiological modeling module is based on a trivariate model.

19. The system of claim 16, wherein the physiological modeling module is based on a cardiovascular short-term regulation model.

20. The system of claim 16, wherein the pre-processing and filtering module filters the physiological measurements from the vital sign sensors to generate a plurality of filtered physiological measurements using at least one of Fourier and wavelet filtering.

21. The system of claim 16, wherein the feature extraction module extracts statistical, spectral, transfer function, long-term trend, and short-term trend features from the plurality of filtered physiological measurements.
